(19) United States
(12) Patent Application Publication
(10) Pub. No.: US 2006/0194824 A1
(43) Pub. Date: Aug. 31, 2006

(54) Compositions containing pyrimidine derivatives as inhibitors of cox-2

(76) Inventors: Martin Rolfe Hill, Research Triangle Park, NC (US); Simon Joseph Holland, Harlow, Essex (GB); Stephanie Lynn Pearson, Harlow, Essex (GB); Kenneth Trevor Yeates, Harlow, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/535,806

(22) PCT Filed: Nov. 21, 2003
(86) PCT No.: PCT/EP03/13344

§ 371 Date: Apr. 11, 2006

(30) Foreign Application Priority Data
Nov. 25, 2002 (GB) 0227443.9

(51) Int. Cl. A61K 31/513; C07D 405/14

(52) U.S. Cl. 514/269; 544/315; 544/316

(57) ABSTRACT

The invention provides a pharmaceutical composition comprising a compound of formula (I) 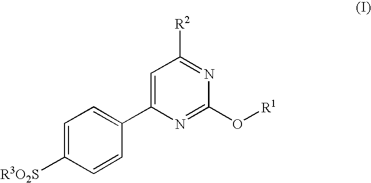 in which the compound is present in solid particles in nanoparticulate form in admixture with one or more pharmaceutically acceptable carriers or excipients.

This invention relates to pyrimidine derivatives, to processes for their preparation, to pharmaceutical compositions containing them and to their use in medicine.

The enzyme cyclooxygenase (COX) has recently been discovered to exist in two isoforms, COX-1 and COX-2. COX-1 corresponds to the originally identified constitutive enzyme while COX-2 is rapidly and readily inducible by a number of agents including mitogens, endotoxin, hormones, cytokines and growth factors. Prostaglandins generated by the action of COX have both physiological and pathological roles. It is generally believed that COX-1 is largely responsible for the important physiological functions such as maintenance of gastrointestinal integrity and renal blood flow. In contrast the inducible form, COX-2, is believed to be largely responsible for the pathological effects of prostaglandins where rapid induction of the enzyme occurs in response to such agents as inflammatory agents, hormones, growth factors and cytokines. A selective inhibitor of COX-2 would therefore have anti-inflammatory, anti-pyretic and analgesic properties, without the potential side effects associated with inhibition of COX-1. We have now found a novel group of compounds which are both potent and selective inhibitors of COX-2.

The invention thus provides the compounds of formula (I) 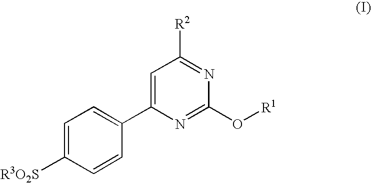
in which:

R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁴R⁵)ₙ and B(CR⁴R⁵)ₙ;

R² is C₁₋₂alkyl substituted by one to five fluorine atoms;

R³ is selected from the group consisting of C₁₋₆alkyl, NH₂ and R⁷CONH;

R⁴ and R⁵ are independently selected from H or C₁₋₆alkyl;

A is an unsubstituted 5- or 6-membered heteroaryl or an unsubstituted 6-membered aryl, or a 5- or 6-membered heteroaryl or a 6-membered aryl substituted by one or more R⁶;

R⁶ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;

B is selected from the group consisting of 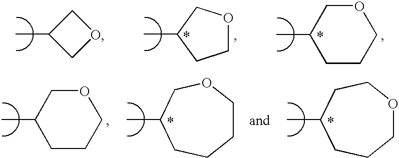
where 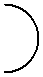
defines the point of attachment of the ring;

R⁷ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl; and

n is 0 to 4.

The term halogen is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

The term 5-membered heteroaryl means a heteroaryl selected from the following:

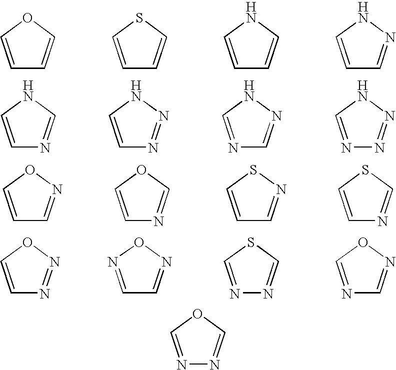

The term 6-membered heteroaryl means a heteroaryl selected from the following:

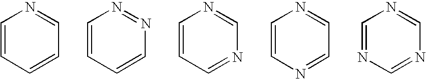

The term 6-membered aryl means:

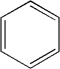

It is to be understood that the present invention encompasses all isomers of the compounds of formula (I) and their pharmaceutically acceptable derivatives, including all geometric, tautomeric and optical forms, and mixtures thereof (e.g. racemic mixtures). In particular when the ring B lacks a plane of symmetry the compounds of formula (I) contain a chiral centre as indicated therein by the asterisk *. Furthermore, it will be appreciated by those skilled in the art that when R⁴ and R⁵ in formula (I) are different the corresponding compounds contain at least one chiral centre, by virtue of the asymmetric carbon atom defined thereby, and that such compounds exist in the form of a pair of optical isomers (i.e. enantiomers).

In one aspect of the invention R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl and B(CR⁴R⁵)ₙ;

In another aspect of the invention R¹ is C₁₋₆alkyl or C₁₋₂alkyl substituted by one to five fluorine atoms. In another aspect R¹ is C₂₋₆alkyl (e.g. n-butyl).

In another aspect of the invention R¹ is C₃₋₁₀cycloalkylC₀₋₆alkyl, such as C₃₋₁₀cycloalkyl (e.g. cyclopentyl or cyclohexyl). In another aspect R¹ is C₃₋₁₀cycloalkylmethyl, such as C₃₋₇cycloalkylmethyl (e.g. cyclopentylmethyl).

In another aspect of the invention R¹ is A(CR⁴R⁵)ₙ.

In another aspect of the invention R² is CHF₂, CH₂F or CF₃. In another aspect R² is CF₃.

In another aspect of the invention R³ is C₁₋₆alkyl, such as C₁₋₃alkyl (e.g. methyl).

In another aspect of the invention R⁴ and R⁵ are independently selected from H or methyl. In another aspect R⁴ and R⁵ are both H.

In another aspect of the invention A is selected from the group consisting of 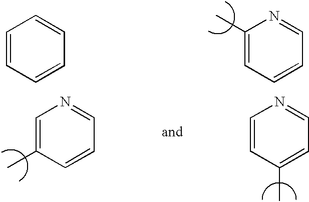
where 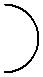
defines the point of attachment of the ring and A is unsubstituted or substituted by one or two R⁶.

In another aspect of the invention R⁶ is selected from the group consisting of halogen (e.g. F), C₁₋₃alkyl (e.g. methyl), C₁₋₃alkyl substituted by one to three fluorine atoms (e.g. CF₃), and C₁₋₃alkoxy (e.g. methoxy).

In another aspect of the invention R⁷ is selected from the group consisting of C₁₋₆alkyl (e.g. ethyl), phenyl and aminomethyl.

In another aspect of the invention n is 1 to 4.

In another aspect of the invention n is 0 to 2 (e.g. 0).

It is to be understood that the invention covers all combinations of particular aspects of the invention as described hereinabove.

Within the invention there is provided one group of compounds of formula (I) (group A) wherein: R¹ is C₁₋₆alkyl (e.g. n-butyl); R² is CF₃; and R³ is C₁₋₆alkyl, such as C₁₋₃alkyl (e.g. methyl).

Within the invention there is provided another group of compounds of formula (I) (group B) wherein: R¹ is C₃₋₁₀cycloalkylC₀₋₆alkyl, such as C₃₋₁₀cycloalkyl (e.g. cyclopentyl or cyclohexyl); R² is CF₃; and R³ is C₁₋₆alkyl, such as C₁₋₃alkyl (e.g. methyl).

Within the invention there is provided another group of compounds of formula (I) (group C) wherein: R¹ is C₃₋₁₀cycloalkylmethyl, such as C₃₋₇cycloalkylmethyl (e.g. cyclopentylmethyl); R² is CF₃; and R³ is C₁₋₆alkyl, such as C₁₋₃alkyl (e.g. methyl).

Within the invention there is provided another group of compounds of formula (I) (group D) wherein: R¹ is A(CR⁴R⁵)ₙ; R² is CF₃; R³ is C₁₋₆alkyl, such as C₁₋₃alkyl (e.g. methyl); R⁴ and R⁵ are independently selected from H or methyl; A is selected from the group consisting of 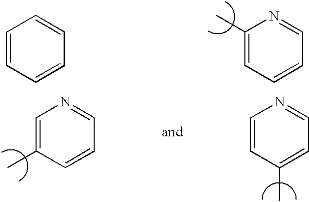
and A is unsubstituted or substituted by one or two R⁶; R⁶ is selected from the group consisting of halogen (e.g. F), C₁₋₃alkyl (e.g. methyl), C₁₋₃alkyl substituted by one to three fluorine atoms (e.g. CF₃), and C₁₋₃alkoxy (e.g. methoxy); and n is 0 to 2 (e.g. 0).

Within group D, there is provided a further group of compounds (group D1) wherein: R¹ is A(CR⁴R⁵)ₙ; R² is CF₃; R³ is methyl; R⁴ and R⁵ are both H; A is selected from the group consisting of 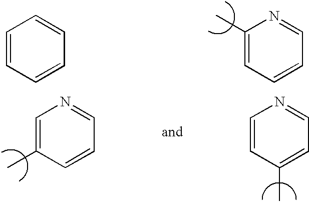
and A is unsubstituted or substituted by one or two R⁶; R⁶ is selected from the group consisting of fluorine, chlorine, methyl, CF₃ and methoxy; and n is 0 or 1.

In a preferred aspect the invention provides the following compounds:

2-(4-fluorophenoxy)-4-[4-(methylsulfonyl)phenyl]-6](trifluoromethyl)pyrimidine;

2-(4-methoxyphenoxy)-4-[4-(methylsulfonyl)phenyl]-6-trifluoromethyl)pyrmidine;

2-butoxy-4-[4-(methylsulfonyl)phenyl]-6-(trifluoromethyl)pyrimidine;

2-[(5-chloropyridin-3-yl)oxy]-4-[4-(methylsulfony)phenyl]-6-(trifluoromethyl)pyrimidine;

2-(cyclohexyloxy)-4-[4-(methylsulfonyl)phenyl]-6-(trifluoromethyl)pyrimidine.

In a more preferred aspect the invention provides the following compound:

2-butoxy-4-[4-(methylsulfonyl)phenyl]-6-(trifluoromethyl)pyrimidine.

Since the compounds of the present invention, in particular compounds of formula (I), are intended for use in pharmaceutical compositions, it will be understood that they are each provided in substantially pure form, for example at least 50% pure, more suitably at least 75% pure and preferably at least 95% pure (% are on a wt/wt basis). Impure preparations of the compound of formula (I) may be used for preparing the more pure forms used in pharmaceutical compositions. Although the purity of intermediate compounds of the present invention is less critical, it will be readily understood that the substantially pure form is preferred as for the compounds of formula (I). Preferably, whenever possible, the compounds of the present invention are available in crystalline form.

When some of the compounds of this invention are allowed to crystallise or are recrysallised from organic solvents, solvent of recrystallisation may be present in the crystalline product. This invention includes within its scope such solvates. Similarly, some of the compounds of this invention may be crystallised or recrystallised from solvents containing water. In such cases water of hydration may be formed. This invention includes within its scope stoichiometric hydrates as well as compounds containing variable amounts of water that may be produced by processes such as lyophilisation. In addition, different crystallisation conditions may lead to the formation of different polymorphic forms of crystalline products. This invention includes within its scope all the polymorphic forms of the compounds of formula (I).

Compounds of the invention are potent and selective inhibitors of COX-2. This activity is illustrated by their ability to selectively inhibit COX-2 over COX-1.

In view of their selective COX-2 inhibitory activity, the compounds of the present invention are of interest for use in human and veterinary medicine, particularly in the treatment of the pain (both chronic and acute), fever and inflammation of a variety of conditions and diseases mediated by selective inhibition of COX-2. Such conditions and diseases are well known in the art and include rheumatic fever, symptoms associated with influenza or other viral infections, such as the common cold; lower back and neck pain; headache; toothache; sprains and strains; myositis; sympathetically maintained pain; synovitis; arthritis, including rheumatoid arthritis; degenerative joint diseases, including osteoarthritis; gout and ankylosing spondylitis; tendinitis; bursitis; skin related conditions, such as psoriasis, eczema, burns and dermatitis; injuries, such as sports injuries and those arising from surgical and dental procedures.

The compounds of the invention are also useful for the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; neuralgia, such as post-herpetic neuralgia and trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of the invention are also useful for the treatment of other conditions mediated by selective inhibition of COX-2.

For example, the compounds of the invention inhibit cellular and neoplastic transformation and metastatic tumour growth and hence are useful in the treatment of certain cancerous diseases, such as colonic cancer and prostate cancer. The compounds of the invention are also useful in reducing the number of adenomatous colorectal polyps and thus reduce the risk of developing colon cancer. The compounds of the invention are also useful in the treatment of cancer associated with overexpression of HER-2/neu, in particular breast cancer.

Compounds of the invention also prevent neuronal injury by inhibiting the generation of neuronal free radicals (and hence oxidative stress) and therefore are of use in the treatment of stroke; epilepsy; and epileptic seizures (including grand mal, petit mal, myoclonic epilepsy and partial seizures).

Compounds of the invention also inhibit prostanoid-induced smooth muscle contraction and hence are of use in the treatment of dysmenorrhoea and premature labour.

Compounds of the invention are also useful in the treatment of liver disease, such as inflammatory liver disease, for example chronic viral hepatitis B, chronic viral hepatitis C, alcoholic liver injury, primary biliary cirrhosis, autoimmune hepatitis, nonalcoholic steatohepatitis and liver transplant rejection.

Compounds of the invention inhibit inflammatory processes and therefore are of use in the treatment of asthma, allergic rhinitis and respiratory distress syndrome; gastrointestinal conditions such as inflammatory bowel disease, Crohn's disease, gastritis, irritable bowel syndrome and ulcerative colitis; and the inflammation in such diseases as vascular disease, migraine, periarteritis nodosa, thyroiditis, aplastic anaemia, Hodgkin's disease, sclerodoma, type I diabetes, myasthenia gravis, multiple sclerosis, sorcoidsis, nephrotic syndrome, Bechet's syndrome, polymyositis, gingivitis, conjunctivitis and myocardial ischemia.

Compounds of the invention are also useful in the treatment of ophthalmic diseases such as retinitis, retinopathies, uveitis and of acute injury to the eye tissue.

Compounds of the invention are also useful for the treatment of cognitive disorders such as dementia, particularly degenerative dementia (including senile dementia, Alzheimer's disease, Pick's disease, Huntington's chorea, Parkinson's disease and Creutzfeldt-Jakob disease), and vascular dementia (including multi-infarct dementia), as well as dementia associated with intracranial space occupying lesions, trauma, infections and related conditions (including HIV infection), metabolism, toxins, anoxia and vitamin deficiency; and mild cognitive impairment associated with ageing, particularly Age Associated Memory Impairment.

Compounds of the invention are also useful in the treatment of disorders ameliorated by a gastroprokinetic agent. Disorders ameliorated by gastroprokinetic agents include ileus, for example post-operative ileus and ileus during sepsis; gastroesophageal reflux disease (GORD, or its synonym GERD); gastroparesis, such as diabetic gastroparesis; and other functional bowel disorders, such as non-ulcerative dyspepsia (NUD) and non-cardiac chest pain (NCCP).

According to a further aspect of the invention, we provide a compound of formula (I) for use in human or veterinary medicine.

According to another aspect of the invention, we provide a compound of formula (I) for use in the treatment of a condition which is mediated by COX-2.

According to a further aspect of the invention, we provide a method of treating a human or animal subject suffering from a condition which is mediated by COX-2 which comprises administering to said subject an effective amount of a compound of formula (I).

According to a further aspect of the invention, we provide a method of treating a human or animal subject suffering from an inflammatory disorder, which method comprises administering to said subject an effective amount of a compound of formula (l).

According to another aspect of the invention, we provide the use of a compound of formula (I) for the manufacture of a therapeutic agent for the treatment of a condition which is mediated by COX-2.

According to another aspect of the invention, we provide the use of a compound of formula (I) for the manufacture of a therapeutic agent for the treatment of an inflammatory disorder.

It is to be understood that reference to treatment includes both treatment of established symptoms and prophylactic treatment, unless explicitly stated otherwise.

It will be appreciated that the compounds of the invention may advantageously be used in conjunction with one or more other therapeutic agents. Examples of suitable agents for adjunctive therapy include a 5HT₁ agonist, such as a triptan (e.g. sumatriptan or naratriptan); an adenosine A1 agonist; an EP ligand; an NMDA modulator, such as a glycine antagonist; a sodium channel blocker (e.g. lamotrigine); a substance P antagonist (e.g. an NK₁ antagonist); a cannabinoid; acetaminophen or phenacetin; a 5-lipoxygenase inhibitor; a leukotriene receptor antagonist; a DMARD (e.g. methotrexate); gabapentin and related compounds; a tricyclic antidepressant (e.g. amitryptilline); a neurone stabilising antiepileptic drug; a mono-aminergic uptake inhibitor (e.g. venlafaxine); a matrix metalloproteinase inhibitor; a nitric oxide synthase (NOS) inhibitor, such as an iNOS or an nNOS inhibitor; an inhibitor of the release, or action, of tumour necrosis factor α; an antibody therapy, such as a monoclonal antibody therapy; an antiviral agent, such as a nucleoside inhibitor (e.g. lamivudine) or an immune system modulator (e.g. interferon); an opioid analgesic; a local anaesthetic; a stimulant, including caffeine; an H₂-antagonist (e.g. ranitidine); a proton pump inhibitor (e.g. omeprazole); an antacid (e.g. aluminium or magnesium hydroxide; an antiflatulent (e.g. simethicone); a decongestant (e.g. phenylephrine, phenylpropanolamine, pseudoephedrine, oxymetazoline, epinephrine, naphazoline, xylometazoline, propylhexedrine, or levo-desoxyephedrine); an antitussive (e.g. codeine, hydrocodone, carmiphen, carbetapentane, or dextramethorphan); a diuretic; or a sedating or non-sedating antihistamine. It is to be understood that the present invention covers the use of a compound of formula (I) in combination with one or more other therapeutic agents.

The compounds of formula (I) are conveniently administered in the form of pharmaceutical compositions. Thus, in another aspect of the invention, we provide a pharmaceutical composition comprising a compound of formula (I) adapted for use in human or veterinary medicine. Such compositions may conveniently be presented for use in conventional manner in admixture with one or more physiologically acceptable carriers or excipients.

As will be appreciated by the person skilled in the art the compounds of the invention may be milled using known milling procedures such as wet milling to obtain a particle size appropriate for tablet formation and for other formulation types. In particular, for those compounds which demonstrate poor bioavailability, finely divided (nanoparticulate) preparations of the compounds of the invention may be prepared by processes known in the art, for example see International Patent Application No. WO 02/00196 (SmithKline Beecham). The invention thus provides, in a further aspect, a pharmaceutical composition wherein the compound of formula (I) is in a finely divided or nanoparticulate form. In particular, the invention provides a pharmaceutical composition comprising 2-butoxy-4-[4-(methylsulfonyl)phenyl]-6-(trifluoromethyl)pyrimidine, in which the compound is present in solid particles in nanoparticulate form in admixture with one or more pharmaceutically acceptable carriers or excipients.

The compounds of formula (I) may be formulated for administration in any suitable manner. They may, for example, be formulated for topical administration or administration by inhalation or, more preferably, for oral, transdermal or parenteral administration. The pharmaceutical composition may be in a form such that it can effect controlled release of the compounds of formula (I).

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative.

Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

As stated above, the compounds of the invention may also be used in combination with other therapeutic agents. The invention thus provides, in a further aspect, a combination comprising a compound of formula (I) together with a further therapeutic agent.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) is used in combination with a second therapeutic agent active against the same disease state the dose of each compound may differ from that when the compound is used alone. Appropriate doses will be readily appreciated by those skilled in the art.

A proposed daily dosage of a compound of formula (I) for the treatment of man is 0.01 mg/kg to 500 mg/kg, such as 0.05 mg/kg to 100 mg/kg, e.g. 0.1 mg/kg to 50 mg/kg, which may be conveniently administered in 1 to 4 doses. The precise dose employed will depend on the age and condition of the patient and on the route of administration. Thus, for example, a daily dose of 0.25 mg/kg to 10 mg/kg may be suitable for systemic administration.

Compounds of formula (I) may be prepared by any method known in the art for the preparation of compounds of analogous structure.

Compounds of formula (I) may be prepared by a process which comprises:

reacting an alcohol R¹OH of formula (II) or a protected derivative thereof with a compound of formula (III) 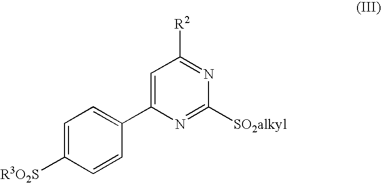
and thereafter and if necessary,

interconverting a compound of formula (I) into another compound of formula (I); and/or

deprotecting a protected derivative of compound of formula (I).

The overall synthesis of a compound of formula (I) is shown in Scheme 1 below in which, R¹ and R² are as defined in formula (I) above unless otherwise stated, R³ is C₁₋₆alkyl; THF is tetrahydrofuran; MTBE is methyl t-butyl ether; and alkyl is a straight or branched chain alkyl group, for example a methyl, ethyl, n-propyl, i-propyl, n-butyl, s-butyl or t-butyl group.

Referring to Scheme 1, the preparation of compounds of formula (I) may conveniently be achieved by the treatment of compounds of formula (III) with an alcohol of formula (II) in the presence of sodium hydride. The reaction is conveniently carried out in a solvent such as THF and at between ambient temperature and reflux.

Conveniently the oxidation shown in Scheme 1 is effected using a monopersulfate compound, such as potassium peroxymonosulfate (known as Oxone™) and the reaction is carried out in a solvent, such as an aqueous alcohol, (e.g. aqueous methanol), and at between −78° C. and ambient temperature.

Alternatively, the oxidation shown in Scheme 1 may be effected using hydrogen peroxide in the presence of catalytic sodium tungstate dihydrate. The reaction may be carried out in a solvent such as acetic acid and at between ambient temperature and reflux (e.g. 50° C.).

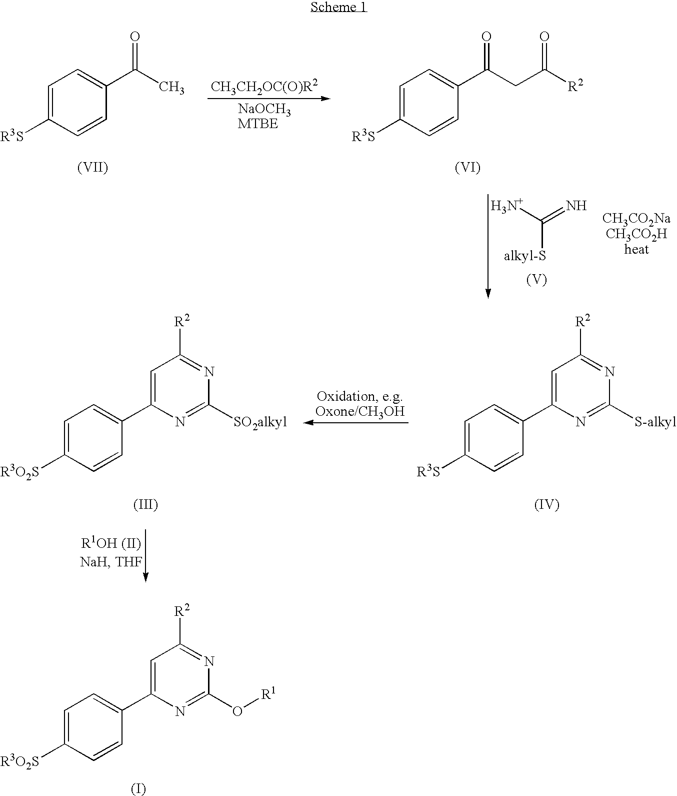

Referring to Scheme 1, the cyclization of diones of formula (VI) to give the corresponding pyrimidines of formula (IV) is conveniently carried out employing a thioronium salt such as a 2-methyl-2-thiopseudourea sulfate and under reflux.

It will be appreciated by those skilled in the art that certain of the procedures described in Scheme 1 for the preparation of compounds of formula (I) or intermediates thereto may not be applicable to some of the possible substituents.

It will be further appreciated by those skilled in the art that it may be necessary or desirable to carry out the transformations described in Scheme 1 in a different order from that described, or to modify one or more of the transformations, to provide the desired compound of formula (I).

In one variation of Scheme 1, compounds of formula (III) wherein R³ is C₁₋₆alkyl or NH₂ may be prepared by oxidising a compound of formula (IV)A: 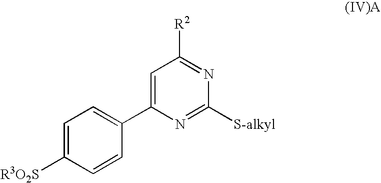
under oxidation conditions described hereinabove. Compounds of formula (IV)A may be prepared according to the general procedures of Scheme 1 by employing sulphonyl derivatives in place of the corresponding sulfide compounds of formulae (VI) and (VII).

It will be appreciated by those skilled in the art that compounds of formula (I) may be prepared by interconversion, utilising other compounds of formula (I) as precursors. Suitable interconversions, such as alkylations, are well known to those skilled in the art and are described in many standard organic chemistry texts, such as ‘Advanced Organic Chemistry’ by Jerry March, fourth edition (Wiley, 1992), incorporated herein by reference. For example, compounds of formula (I) wherein R¹ is C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkane, A(CR⁴R⁵)ₙ (with the proviso that n is not zero) and B(CR⁴R⁵)ₙ may be prepared by alkylating the corresponding compound of formula (I) wherein R¹ is H.

Acylation of compounds of formula (I) wherein R³ is NH₂, to provide compounds of formula (I) wherein R³ is R⁷CONH, may be carried out by conventional means, for example by employing conventional acylating agents such as those described in ‘Advanced Organic Chemistry’, pp 417-424, incorporated herein by reference.

As will be appreciated by those skilled in the art it may be necessary or desirable at any stage in the synthesis of compounds of formula (I) to protect one or more sensitive groups in the molecule so as to prevent undesirable side reactions. The protecting groups used in the preparation of compounds of formula (I) may be used in conventional manner. See, for example, those described in ‘Protective Groups in Organic Synthesis’ by Theodora W Green and Peter G M Wuts, second edition, (John Wiley and Sons, 1991), incorporated herein by reference, which also describes methods for the removal of such groups.

Alcohols of formula (II) are either known compounds or may be prepared by literature methods, such as those described in ‘Comprehensive Organic Transformations: a guide to functional group preparations’ by Richard Larock (VCH, 1989), incorporated herein by reference.

Thioronium salts of formula (V) are either known compounds or may be prepared by literature methods, such as those described in A H Owens et al, Eur J Med Chem, 1988, 23(3), 295-300, incorporated herein by reference.

Acetophenones of formula (VII) are either known compounds or may be prepared by conventional chemistry.

Certain intermediates described above are novel compounds, and it is to be understood that all novel intermediates herein form further aspects of the present invention. Compounds of formulae (III) and (IV) are key intermediates and represent a particular aspect of the present invention.

Solvates (e.g. hydrates) of a compound of the invention may be formed during the work-up procedure of one of the aforementioned process steps;

The Intermediates and Examples that follow illustrate the invention but do not limit the invention in any way. All temperatures are in ° C. Flash column chromatography was carried out using Merck 9385 silica. Solid Phase Extraction (SPE) chromatography was carried out using Varian Mega Bond Elut (Si) cartridges (Anachem) under 15 mmHg vacuum. Thin layer chromatography (Tlc) was carried out on silica plates. In addition to those already defined, the following abbreviations are used: Me, methyl; Ac, acyl; DMSO, dimethylsulphoxide; TFA, trifluoroacetic acid; DME, dimethoxyethane; DCM, dichloromethane; NMP, N-methyl pyrrolidone; and MTBE, methyl t-butyl ether.

### INTERMEDIATE 1

### 4,4,4-Trifluoro-1-[4-(methylthio)phenyl]butane-1,3-dione

To a solution of ethyl trifluoroacetate (7.95 ml, 1.1 eq) in MTBE (125 ml) was added dropwise 25% sodium methoxide in methanol (16 ml, 1.2 eq). 4-Methylthioacetophenone (Aldrich, 10 g, 0.06 mol) was added portionwise and the mixture stirred at ambient temperature overnight. 2N Hydrochloric acid (40 ml) was added cautiously and the organic phase separated, washed with brine and dried (Na₂SO₄) to give an orange solid. The orange solid was recrystallised from hot isopropanol to give the title compound as a yellow crystalline solid (11.25 g, 71%).

MH-261

### INTERMEDIATE 2

### 2-(Methylthio)-4-[4-(methylthio)phenyl]-6-(trifluoromethyl)pyrimidine

To a mixture of 4,4,4-trifluoro-1-[4-(methylthio)phenyl]butane-1,3-dione (5 g) and 2-methyl-2-thiopseudourea sulfate (5.1 g, 0.98 eq) in acetic acid (100 ml) was added sodium acetate (3 g, 2 eq) and heated under reflux for 8 h. The mixture was concentrated in vacuo and water (100 ml) added to give a solid, which was isolated by filtration to give the title compound as a yellow solid (5.8 g, quantitative).

MH+317

### INTERMEDIATE 3

### 2-(Methylsulfonyl)-4-[4-(methylsulfonyl)phenyl-6-(trifluoromethyl)pyrimidine

To a solution of 2-(methylthio)-4-[4-(methylthio)phenyl]-6-(trifluoromethyl)pyrimidine (5.78 g) in MeOH (500 ml) was added a solution of OXONE™ (Aldrich, 56.23 g, 5 eq) in water (200 ml). The mixture was stirred at ambient temperature overnight, concentrated in vacuo and the residue partitioned between water and ethyl acetate (2×100 ml). The combined organic phases were dried and concentrated in vacuo to an off-white solid which was triturated with hot isopropanol to give the title compound as a white solid (5.6 g, 80%).

MH+381

Tlc SiO₂ Ethyl acetate:cyclohexane (1:1) Rf 0.45

### EXAMPLE 1

### 2-(4-Fluorophenoxy)-4-[4-(methylsulfonyl)phenyl]-6-(trifluoromethyl)pyrimidine

To a stirred solution of 4-fluorophenol (37 mg, 0.33 mmole) in dry tetrahyrofuran (10 ml) was added, under an atmosphere of nitrogen, sodium hydride (60% dispersion in oil, 13 mg, 0.33 mmole) and the resulting mixture stirred at 20 for 30 min. To the stirred reaction mixture was added 2-(methylsulfonyl)-4[4-(methylsulfonyl)phenyl]-6-trifluoromethyl)pyrimidine (114 mg, 0.33 mmole) in a single portion, and stirring was continued for 2 h. The solvent was evaporated, and the residue partitioned between dichloromethane and 2N sodium hydroxide. The dried organic phase was evaporated to dryness. The residue was purified on a silica gel SPE cartridge eluting with chloroform to afford the title compound as a colourless solid (99 mg, 80%).

MH+413.

### EXAMPLES 2 TO 10

Examples 2 to 10, as shown in Table 1 that follows, were prepared in the manner described for Example 1.

TABLE 1

| | |
|---|---|
| | (I) |
| 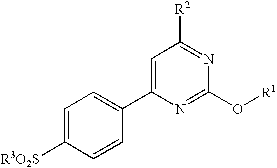 | |

| Ex | R¹ | R² | R³ | MS |
|---|---|---|---|---|
| 2 | 3,4-difluorophenyl | CF₃ | CH₃ | MH+ 431 |
| 3 | 4-methoxyphenyl | CF₃ | CH₃ | MH+ 425 |
| 4 | 4-fluorobenzyl | CF₃ | CH₃ | MH+ 427 |
| 5 | 4-bromophenyl | CF₃ | CH₃ | MH+ 474 |
| 6 | 4-methylphenyl | CF₃ | CH₃ | MH+ 409 |
| 7 | 5-chloropyridin-3-yl | CF₃ | CH₃ | MH+ 431 |
| 8 | cyclohexyl | CF₃ | CH₃ | MH+ 401 |
| 9 | cyclopentylmethyl | CF₃ | CH₃ | MH+ 401 |
| 10 | n-butyl | CF₃ | CH₃ | MH+ 375 |

### EXAMPLE 11

### 2-Butoxy-4-[4-(methylsulfonyl)phenyl]-6-(trifluoromethyl)pyrmidine

Sodium methoxide (6.6 kg of a 30% w/w solution in methanol) was added over at least 30 min to a solution of 4-(methylthio)acetophenone (5.0 kg) and methyl trifluoroacetate (4.25 kg) in tert-butylmethylether (40 L) at 40±3° C. The solution was heated at 40±3° C. for at least 3 h. Acetic acid (55 L) was added, followed by S-methyl 2-thiopseudourea sulfate (5.45 kg) and the mixture concentrated to ca. 45L. The mixture was heated at about 110° C. for at least a further 8 h (overnight) then acetic acid (20 L) was added before cooling to 50±3° C. A solution of sodium tungstate dihydrate (0.2 kg) in water (2.5 L) was added, followed by hydrogen peroxide (20.7 kg of 30% w/v solution), which was added over at least 3 h, maintaining the temp at ca. 50°. The mixture is heated at ca. 50° C. for at at least 12 h before cooling to 20±3° C. A solution of sodium sulphite (3.45 kg) in (28 L) was then added over at least 30 min whilst maintaining the temperature at 20±3°. The mixture was aged at 20±3° C. for ca. 1 h and 2-methylsulfonyl)-4-[4-(methylsulfonyl)phenyl]-6-(trifluoromethyl)pyrimidine collected by filtration, washed with water (3×15 L) and dried at up to 60° in vacuo.

Yield 9.96 kg, 90% of theory.

A suspension of 2-(methylsulfonyl)-4-[4-(methylsulfonyl)phenyl]-6-(trifluoromethyl)pyrimidine (525 g) in n-butanol (5.25 L) was treated with potassium carbonate (210 g) at 20±50° C. The mixture was heated to 50±5° C. overnight until the reaction was complete by HPLC. Acetic acid (1.57 L) was added dropwise, to control any gas evolution, keeping the temperature at 50±5° C. Water (3.67 L) was. then added over 30 min keeping the temperature at 50±5° C. to allow full crystallisation to occur. The slurry was then cooled to 20-25° C. and aged for at least 1 hour. The resulting product was then filtered under vacuum and washed with a mixture of n-butanol (787 mL), acetic acid (236 mL), and water (551 mL) followed by water (2×1.57 L). The product was then dried at up to ca50° C. under vacuum to yield the title compound. Yield, 457 g, 88.4% of theory. The title compound was found to be identical to that of Example 10.

¹H NMR (CDCl₃) δ: 8.33(2H, d, para-di-substituted CH); 8.11(2H, d, para-di-substituted CH); 7.70(1H, s, aromatic CH); 4.54(2H, t, butyl CH₂); 3.12(3H, s, sulphone CH₃); 1.88(2H, m, butyl CH₂); 1.55(2H, m, butyl CH₂); 1.01 (3H, t, butyl CH₃).

### EXAMPLE 12

### Pharmaceutical Composition Comprising 2-Butoxy-4-[4-(methylsulfonyl)phenyl]-6-(trifluoromethyl)pyrimidine in Nanoparticulate Form

A 2 kg batch of an aqueous suspension containing 10% w/w of 2-butoxy-4-[4-(methylsulfonyl)phenyl]-6-(trifluoromethyl)pyrimidine (Example 11) and 3.6% w/w of hydroxypropylmethylcellulose was passed through a Dena DM-100 bead mill. The single 100 ml chamber fabricated from Nylacast Nylube was used in a recirculation configuration with the chamber containing 86% by volume of yttrium stabilised zirconium oxide beads (Tosoh, Japan). The batch was processed using a single bead size, a 0.4 mm diameter bead sample. The batch was processed for 165 minutes. The yield was 97.0% w/w. To the finely milled suspension was added 15% w/w mannitol and the resulting suspension subsequently spray-dried to yield the title pharmaceutical composition.

Grinding media contamination levels in the spray-dried powder (Example 12) were <2 ppm zirconium (Zr) and <1 ppm yttrium (Y).

The reconstituted spray dried powder product had a median particle size of 2.4 microns as measured by laser diffraction size analysis using a Malvern Mastersizer S laser diffraction unit using Fraunhofer evaluation.

### EXAMPLE 13

### Pharmaceutical Composition Comprising 2-Butoxy-4-[4-(methylsulfonyl)phenyl]-6-(trifluoromethyl)pyrimidine in Nanoparticulate Form

A 5.2 kg batch of an aqueous suspension containing 20% w/w of 2-butoxy-4-[4-(methylsulfonyl)phenyl]-6-(trifluoromethyl)pyrimidine (Example 11), 1.0% w/w of hydroxypropylmethylcellulose, 0.2% w/w of sodium lauryl sulphate and 10% w/w mannitol was passed through a Nylacast twin chamber bead mill. Each of the two 1000 ml chambers fabricated from Nylacast Nylube was used In a recirculation configuration with the chamber containing 86% by volume of yttrium stabilised zirconium oxide beads (Tosoh, Japan). The batch was processed using two bead sizes, a 0.8 mm diameter bead sample, and a 0.4 mm bead sample. The batch was processed for 120 minutes. The yield was 95.0% w/w. The resulting suspension was subsequently spray-dried to yield the title pharmaceutical composition.

Grinding media contamination levels in the spray-dried powder (Example 13) were 3 ppm zirconium (Zr) and <1 ppm yttrium (Y).

The reconstituted spray dried powder product had a median particle size of 0.34 microns and a 90% volume particle size of 1.02 microns as measured by laser diffraction size analysis using a Malvern Mastersizer S laser diffraction unit using Fraunhofer evaluation.

1. A pharmaceutical composition comprising a compound of formula (I) 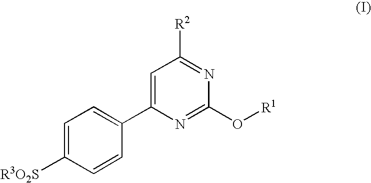
wherein:
R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁴R⁵)ₙ and B(CR⁴R⁵)ₙ;
R² is C₁₋₂alkyl substituted by one to five fluorine atoms;
R³ is selected from the group consisting of C₁₋₆alkyl, NH₂ and R⁷CONH;
R⁴ and R⁵ are independently selected from H or C₁₋₆alkyl;
A is an unsubstituted 5- or 6-membered heteroaryl or an unsubstituted 6-membered aryl, or a 5- or 6-membered heteroaryl or a 6-membered aryl substituted by one or more R⁶;
R⁶ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is selected from the group consisting of 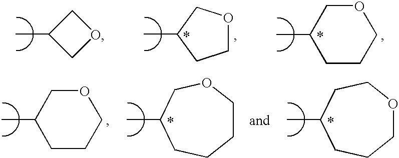
where 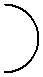
defines the point of attachment of the ring;
R⁷ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl; and
n is 0 to 4
in which the compound is present in solid particles in nanoparticulate form in admixture with one or more pharmaceutically acceptable carriers or excipients.

2. A pharmaceutical composition comprising 2-butoxy-4-[4-(methylsulfonyl)phenyl]-6-(trifluoromethyl)pyrimidine in which the compound is present in solid particles in nanoparticulate form in admixture with one or more pharmaceutically acceptable carriers or excipients.
